(19) United States
(12) Patent Application Publication
(10) Pub. No.: US 2017/0197052 A1
(43) Pub. Date: Jul. 13, 2017

(54) Medical mask for EGD, ERCP and bronchoscopy

(71) Applicants: Angela Tylka, Chicago, IL (US); David J. Roth, Chicago, IL (US)

(72) Inventors: Angela Tylka, Chicago, IL (US); David J. Roth, Chicago, IL (US)

(21) Appl. No.: 14/992,571

(22) Filed: Jan. 11, 2016

(51) Int. Cl. A61M 16/06; A61B 90/16

(52) U.S. Cl. CPC A61M 16/06; A61B 90/16; A61M 2202/0208

(57) ABSTRACT

An improved medical gas supply mask adapted to allow insertion of a broncoscope or endoscope into a patient's mouth without removing the mask. The mask's gas supply portal is located below the mouth instead of directly below the nose. The mask has an attached, typically extendable, tubular bite block that has an outer orifice on the outside of the mask. The surgeon can thus manipulate a scope tube or other instrument into the esophagus or trachea as desired while still providing oxygen to the patient. The mask includes small closed side-ports to receive items like a CO2 monitor tube and the like. Instruments up to approximately 4 cm may be inserted.

### BACKGROUND

Field of the Invention

The present invention relates generally to medical masks and more particularly to a medical mask that allows endoscopic and broncoscopic instruments to be used without removing the mask.

Description of the Prior Art

Sterile, disposable oxygen masks are in common use in hospitals and other medical facilities. These masks typically cover the nose and mouth (See FIG. 1A and FIG. 2A) and are strapped around the head with an elastic strap. With this type of mask, oxygen (or other gas) is typically supplied through a portal located under the nose; however, the patient also breaths air from around the sides of the mask since the flow of oxygen is of less volume that the volume of gas needed to breath. These masks cannot usually be used for procedures that require access to the mouth or nose. In particular, they cannot be used for Esophagogastroduodenoscopy (EGD), Endoscopic Retrograde Cholangiopancreatography (ERCP) and Bronchoscopy. All of these procedures require access to the mouth with medical instruments. Nevertheless, it is critical for the patient to receive oxygen during these procedures. An improved mask is needed.

EGD is a procedure usually performed by a gastroenterologist to diagnose structural or functional abnormalities of the esophagus, stomach or duodenum. A long, flexible lighted tube called an endoscope is inserted through the patient's mouth and throat and into the esophagus. Instruments can be inserted through the tube to perform procedures such as biopsies.

ERCP is a procedure that uses an endoscope in combination with radiography to diagnose diseases of the pancreatic and bile ducts. Again, the endoscope is inserted through the mouth and throat into the esophagus and into the duodenum to the pancreatic ducts and/or bile ducts.

Bronchoscopy is a procedure to view the airways and lungs using a lighted scope. The broncoscope tube (also spelled bronchoscope) is inserted through the patient's mouth or nose and into the trachea and on to the lungs. The tube is usually flexible, but may also be rigid in some cases.

All of these and other similar procedures require access to the mouth. This is not possible with a standard, disposable oxygen mask because typically the gas entry port and bag are located in a position that is in front of the mouth. Yet to forego oxygen during these procedures can put the patient in jeopardy. It would be extremely advantageous to have a flexible, disposable oxygen mask that allows access to the mouth by tube-like instruments such as endoscopes and broncocsopes.

### SUMMARY OF THE INVENTION

The present invention relates to an improved flexible, disposable oxygen mask that has been adapted to allow insertion of an endoscope, broncoscope or other instrument into the patient's mouth without removing the mask. The present invention resembles the prior art flexible mask except that the lower part of the mask extends outwardly to almost the same extent as the nose part, and the oxygen supply portal is located below or to the side of the mouth, between the mouth and chin, instead of directly below the nose. Alternatively, one or more slits can be cut in the flexible mask material proximate to the location of the mouth, or a diaphragm can be placed over the mouth area. In an alternative embodiment, an insertion tube with a bite block is located in the mouth area of the mask. This tube can extend past the patient's teeth into the mouth, and can be extendable for correct fit. The slits, diaphragm or open end of the insertion tube can remain substantially closed when not being used allowing very little oxygen to leak out. In embodiments with a tube and bite block, holes in the tube allow breathing through the mouth as well as the nose.

When a tube or other instrument is inserted, the slit or diaphragm receives the tube and closes around it again without leaking much oxygen. The surgeon can thus manipulate the scope tube into the esophagus or trachea as desired while still providing oxygen to the patient. The mask includes small side-ports that contain one-way valves to act as non re-breathers for exhalation, and a port to receive items such as a CO2 monitor tube and the like. An embodiment of the mask has an adjustable bite block for the patient's teeth.

### DESCRIPTION OF THE FIGURES

Attention is now directed to several figures that illustrate features of the present invention.

FIG. 1A is a side view of a prior art flexible oxygen mask.

FIG. 1B is a side view of an embodiment of the present invention.

FIG. 2A is a front view of the prior art mask of FIG. 1A.

FIG. 2B is a front view of the embodiment of the invention of FIG. 1B.

FIG. 3 shows a front view of an embodiment of the invention with a diaphragm.

FIG. 4 shows a sectional view of an embodiment with a bite block extended.

FIG. 5 shows a non-sectional side view of the embodiment of FIG. 4 with the bite block shown with hidden lines and retracted.

FIG. 6 is a front view of the embodiment of FIGS. 4-5.

Several drawings and illustrations have been provided to aid in understanding the present invention. The scope of the present invention is not limited to what is shown in the figures.

### DESCRIPTION OF THE PREFERRED EMBODIMENTS

The present invention is an improvement over prior art flexible oxygen masks used in hospitals and other medical facilities. The present invention allows insertion of an endoscope, broncoscope or other instrument into a patient's mouth without removing the mask. In some embodiments, an adjustable bite block accommodates the patient's teeth.

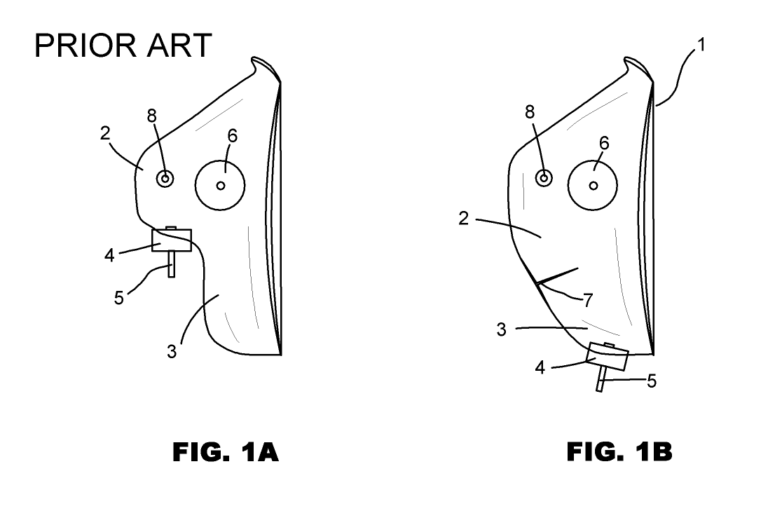

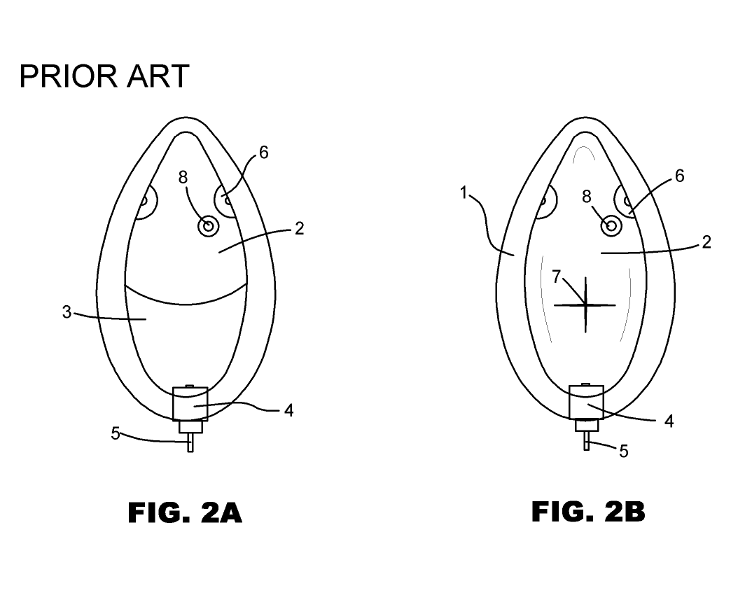

FIG. 1A shows a side view of a prior art mask 1; while FIG. 2A shows a front view of the prior art mask. The prior art mask is characterized by an upper protruding nose section 2 and a recessed lower mouth section 3. A gas entry portal 4 attaches to the mask 1 just below the patient's nose on the lower part of the nose section 2. Oxygen is supplied through a tube nipple 5, while a ventilator bag is usually attached to the lower part of the portal. One or more non rebreather or exhalation ports 6 can usually be found on each side of the nose portion of the mask. These are one-way valves pass gas out of the mask upon exhaling. There can also be an auxiliary port 8 that can be used for CO2 monitors and the like. It can be seen that any access to the patient's mouth, even if there were a hole in the mask, is blocked by the gas entry portal 4.

FIG. 1B shows a side view, while FIG. 2B shows a front view of an embodiment of the present invention. The present invention is characterized by the mask 1 not having a recessed lower mouth region. In fact, the lower mouth region 3 protrudes outward to almost the extent of the upper nose region 2. The gas entry portal 4 and oxygen entry nipple 5 have been moved to a location on the bottom of the mouth region 3 between the mouth and chin. In this embodiment, a single or multiple slit such as a crossed, double slit 7 is located on the mouth region 3 in proximity to the patient's mouth or proximate the patient's nose. The double slit 7 includes a vertical cut and a horizontal cut at right angles that form a cross. A diaphragm may also be used. The slit in the flexible material seals tightly around an inserted instrument and closes almost completely when nothing is inserted. The size of an inserted instrument or tube can be up to approximately 4 cm or slightly larger. This prevents all but a tiny leakage of oxygen (this type of mask is not sealed, and the patient also draws air in around the edges of the mask—as opposed to a ventilator mask that forces the patient to breath and must be sealed). While the preferred embodiment uses one or more slits or a diaphragm, any orifice that allows entry of a medical instrument, and does not leak a substantial amount of oxygen, is within the scope of the present invention. The present invention also includes the non-rebreather port 6 and auxiliary port 8 similar to the prior art mask in approximately the same position.

Embodiments of the mask of the present invention allow easy insertion of an endoscope, broncoscope or other medical instrument into the patient's mouth without removal of the mask or interruption of the flow of oxygen. The instrument can then be maneuvered into the esophagus or trachea as desired. Instruments or tubes up to around 4 cm may be inserted. Auxiliary tubes such as a CO2 monitor can be inserted into the auxiliary port 8.

It should be noted that while the preferred embodiment has a double slit in the shape of a cross (two slits at right angles), a single slit or multiple slits in any configuration, or a diaphragm is within the scope of the present invention. Also, while the preferred embodiment locates the gas entry portal near or below the chin of the patient, any location that does not obstruct the mouth is within the scope of the present invention. Finally, while the mask of the present invention is typically used with oxygen, any gas is within the scope of the present invention.

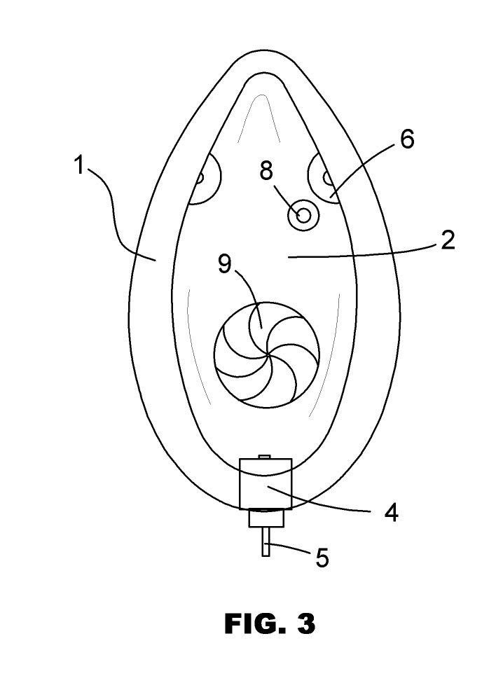

FIG. 3 shows an alternative embodiment of the invention that uses a diaphragm 9 located proximate the patient's mouth. The diaphragm 9 performs the same function as the slit or slits 7 of other embodiments. A diaphragm is typically more versatile than a slit in that it can easily adjust to any diameter of inserted tube or instrument. Other features of this embodiment are similar to those of the embodiments of FIGS. 1B and 2B.

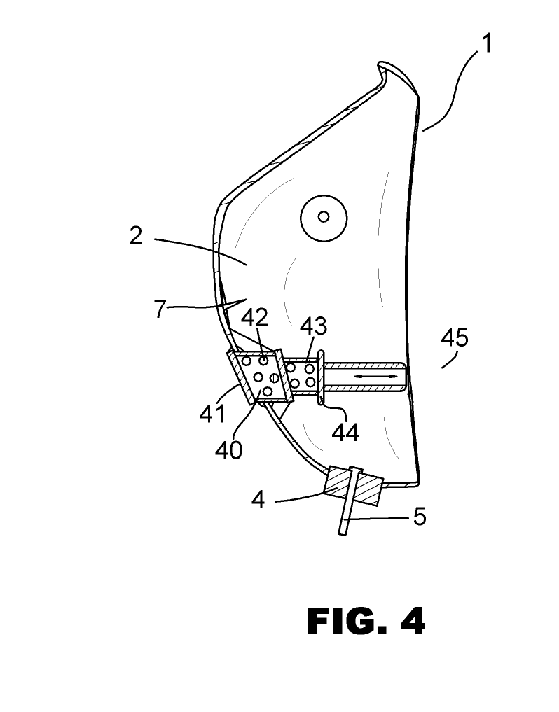

FIG. 4 shows a sectional view of an alternative embodiment of the present invention. A tube 40 extends through the front of the mask 1 below the nose region 2 in proximity to the patient's mouth. The tube 40 includes a bite block 43 for the patient's teeth that can optionally telescope into the tube 40 to adjust for proper size. The bite block 43 has a rim 44 that fits inside the patient's teeth. An optional extension tube 45 can extend toward the patient's throat. The tubes can be locked with a twist lock or any other method of securing the extension bite block section 43 to the main tube 40. Holes 42 can be provided in the tube 40 in the interior of the mask 1. The front end 41 of the main tube 40 is adapted to allow insertion of instruments into the patient's throat through the tubes while the patient breaths both through the nose and through the tube 40. The end 41 is typically flush with the surface of the mask.

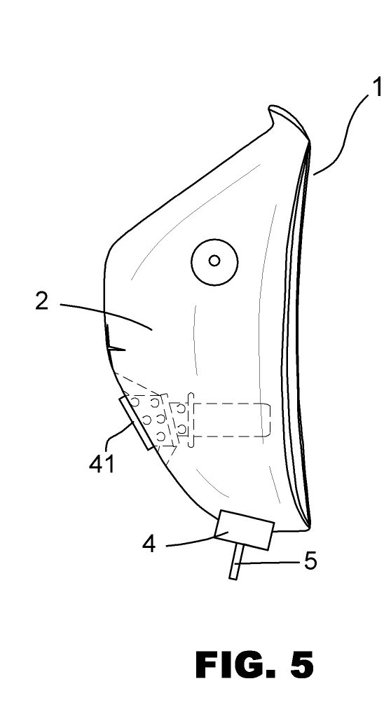

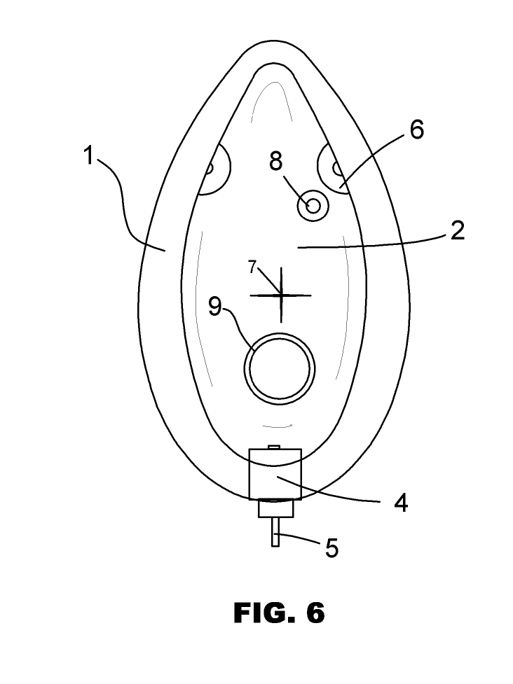

FIG. 5 shows a non-sectional side view of the embodiment of FIG. 4 with the bite block retracted and shown with hidden lines. It can be seen that the tube 40 extends through the front of the mask in the area of the patients mouth. FIG. 6 is a front view of the embodiment of FIGS. 4-5.

The tubes 40 and 43 and the rim 44 are typically made of substantially rigid material such as a rigid plastic. However, the bite block 43, can optionally be made from a softer, more spongy material such as a compressible soft plastic or a silicone rubber so that it will compress somewhat if the patient bites down hard.

In all of the embodiments described, the external orifice 41 of the tube 40 tube can initially be covered with a removable thin cover such as a thin piece of tape or gauze.

Several descriptions and illustrations have been presented that aid in understanding the present invention. One with skill in the art will realize that numerous changes and variations may be made without departing from the spirit of the invention. Each of these changes and variations is within the scope of the present invention.

1. A medical gas supply mask having a top, bottom, and edges, of a type supplying less gas flow than is needed to breath, comprising a flexible, non-sealing mask body that allows gas to pass around the edges of the mask with a gas supply port located on said mask in a position near the bottom of the mask and a telescoping bite block extending inwardly from a position on the mask to a second position arranged proximate a wearer's mouth when in use; the bite block adapted to be inserted into the wearer's mouth, the telescoping bite block comprising a pair of hollow concentric tubes, an inner tube and an outer tube; the inner tube having a substantially cylindrical surface with a rim adapted to fit into the wearer's mouth; the outer tube having an open proximal end accessible from outside the mask facilitating insertion of a medical instrument; the inner tube telescoping to a plurality of use positions with respect to the outer tube.

2. (canceled)

3. The medical gas supply mask of claim 1 wherein the bite block is removable from the mask.

4. The medical gas supply mask of claim 1 wherein the open proximal end of the tube is supplied with a thin removable cover.

5. The medical gas supply mask of claim 1 wherein the mask is made of silicone rubber.

6. The medical gas supply mask of claim 1 wherein the inner tube of the bite block contains a plurality of holes in the substantially cylindrical surface.

7. The medical gas supply mask of claim 1 wherein the bite block is adapted to receive a medical instrument of up to approximately 4 cm in diameter.

8. A flexible medical gas supply mask having a top, a bottom, and edges comprising:
a flexible non-sealing mask body of a type that allows gas to pass around the edges of the mask, the mask having an upper nose portion and a lower mouth portion, the upper nose and lower mouth portions both protruding outward to approximately equal extent;
a gas supply port located on the bottom of the mask;
an extendable bite block comprising an outer tube and an inner tube that telescopes in the outer tube, the outer tube attached to the mask at a point proximate a location of a wearer's mouth when in use, the bite block adapted to be inserted into the wearer's mouth and adjusted in length by telescoping to a plurality of fixed positions to fit the wearer's mouth and to allow instruments to be passed through the bite block;
the bite block adapted to receive a wearer's teeth.

9. The flexible medical gas supply mask of claim 8 wherein the mask is made of silicone rubber.

10. The flexible medical gas supply mask of claim 8 wherein the gas supply port is constructed to receive a supply of oxygen gas.

11. The flexible medical gas supply mask of claim 8 wherein the inner tube has a plurality of holes.

12. The flexible medical gas supply mask of claim 8 wherein the mask further includes at least one auxiliary port located on the upper nose portion.

13. A method of supplying a medical gas mask having a top, bottom, and edges, the mask being of flexible and non-sealing used to allow gas to flow around the mask edges that allows entry of endoscopic instruments into a wearer's mouth without removing the mask comprising:
providing a flexible, non-sealing mask adapted to allow air to enter the mask around the edges with a gas supply port located on the bottom of the mask;
providing said flexible, non-sealing mask with a telescoping attached tubular bite block adapted to allow insertion of an endoscopic instrument into the wearer's throat through the tubular bite block, the tubular bite block comprising an outer tube attached to the mask and an inner tube constructed to slide in the outer tube to a plurality of fixed positions.

14. The method of claim 13 wherein the tubular bite block has an external orifice adapted to allow insertion of instruments up to approximately 4 cm in diameter into the wearer's throat.

15-16. (canceled)

17. The method of claim 13 wherein the mask has at least one non-rebreather port.

18. The method of claim 13 wherein the gas supply port is constructed to receive a supply of oxygen gas.

19. The method of claim 13 wherein the inner tube has a plurality of holes.

20. The medical gas supply mask of claim 1 wherein the outer tube has a plurality of holes.
